SEQUENCE LISTING (6 sequences)

SEQ ID NO: 1 - protein, 187 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKE

SEQ ID NO: 2 - protein, 188 aa, Unknown
  Megabat
ATRRYYLGAVELSWDYMQSELLSELHMDTRFPPEVPRSFPFNTSVIYKKTVFVEFTDHLFNTAKPRPPWMGLLGPTIRAEVSDTVVITLKNMASHAVSLHAVGVSYWKASEGAQYEDQTSQREKEDDKVIPGDSHTYVWEVLKENGPMASDPPCLTYSYFSHVDLVKDLNAGLIGTLLVCREGSLAKE

SEQ ID NO: 3 - protein, 336 aa, Homo sapiens
ATRRYYLGAVELSWDYMQSSLLELPVDARFPPRVPKSFPFNTSVVYKKTVFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPPCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLR

SEQ ID NO: 4 - protein, 336 aa, Homo sapiens
ATRRYYLGAVELSWDYMQSSLGELPVDARFPPRVPKSFPFNTSVVYKKTVFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPPCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLR

SEQ ID NO: 5 - protein, 1457 aa, Homo sapiens
MQIELSTCFFLCLLRFCFSATRRYYLGAVELSWDYMQSSLLELPVDARFPPRVPKSFPFNTSVVYKKTVFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPPCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 1457 aa, Homo sapiens
MQIELSTCFFLCLLRFCFSATRRYYLGAVELSWDYMQSSLGELPVDARFPPRVPKSFPFNTSVVYKKTVFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPPCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2024/0254197 A1
(43) Pub. Date: Aug. 1, 2024

(54) Engineered Human FVIII with Enhanced Secretion Ability and Clotting Activity

(71) Applicant: Sichuan Real&Best Biotech Co., Ltd., Chengdu (CN)

(72) Inventors: Biao Dong, Chengdu (CN); Bo Zhang, Chengdu (CN); Jingya Ye, Chengdu (CN); Lin Xiao, Chengdu (CN); Zhaoyue Zheng, Chengdu (CN); Li Yang, Chengdu (CN); Yu Liu, Chengdu (CN)

(73) Assignee: Sichuan Real&Best Biotech Co., Ltd., Chengdu (CN)

(21) Appl. No.: 17/774,450

(22) PCT Filed: Feb. 11, 2022
(86) PCT No.: PCT/CN2022/075976

§ 371 Date: May 4, 2022

(30) Foreign Application Priority Data
Nov. 25, 2021 (WO) PCT/CN2021/133004

(51) Int. Cl. C07K 14/755; A61K 38/00; C12N 15/86

(52) U.S. Cl. CPC C07K 14/755; C12N 15/86; A61K 38/00; C12N 2750/14143

(57) ABSTRACT

The present invention relates to an engineered human factor VIII polypeptide, which includes at least two substituted amino acids in A1 domain of hFVIII. In some embodiments, the substituted amino acids comprise L50V and L152P. In some embodiments, the substituted amino acids further comprise one or more of amino acid substitutions selected from the group consisting of D20S, G22L, I61T, D115E, F129I, G132D, Q139E, and L159F. The present invention also relates to engineered hFVIII polypeptide encoding nucleic acid fragment, an expression vector or a rAAV vector contains such nucleic acid fragment, and a method of using engineered hFVIII polypeptide to treat hemophilia A patients.

### CROSS-REFERENCE TO RELATED APPLICATIONS

This application claims priority to PCT Application No. PCT/CN2021/133004, filed on Nov. 25, 2021, the contents of which are incorporated herein by reference in their entirety.

### BACKGROUND OF THE INVENTION

Human factor VIII is a protein encoded by the F8 gene located on the X chromosome and is composed of 2351 amino acids. Defects in F8 gene result in the absence or deficiency of the factor VIII it encodes. Hemophilia A (HA) is a hereditary bleeding disorder caused by factor VIII deficiency, which includes deficiency in clotting activity caused by production of defective factor VIII, by inadequate or no production of factor VIII, or by partial or total inhibition of factor VIII by inhibitors. Due to factor VIII deficiency, the blood of HA patients cannot clot properly to control bleeding.

The common treatment for HA is replacement therapy. Concentrates of factor VIII are slowly dripped or injected into a vein of HA patients. These infusions help replace the factor VIII that is missing or low in a patient. However, this replacement therapy may generate inhibitors of the injected or acquired factor VIII, leading to the failure of this replacement therapy.

### SUMMARY OF THE INVENTION

The present invention provides engineered human FVIII polypeptides, FVIII encoding nucleic acids, and FVIII expression vectors. FVIII containing pharmaceutical compositions, as well as methods of using thereof to address the need in the field, such as treating hemophilia A.

In one aspect, the present invention provides an engineered human factor VIII (hFVIII) polypeptide comprising at least two substituted amino acids in A1 domain of hFVIII.

In some embodiments, the substituted amino acids comprise L50 and L152 in the A1 domain.

In some embodiments, the substituted amino acids include L50V and L152P in the A1 domain.

In some embodiments, the substituted amino acids further comprise one or more of amino acid substitutions selected from the group consisting of D20, G22, I61, D115, F129, G132, Q139, and L159 in the A1 domain.

In some embodiments, the substituted amino acids further include one or more of amino acid substitutions selected from the group consisting of D20S, G22L, I61T, A115E, F129I, G132D, Q139E, and L159F in the A1 domain.

In some embodiments, the substituted amino acids comprise D20S, L50V, and L152P.

In some embodiments, the substituted amino acids comprise D20S, G22L, L50V, and L152P.

In some embodiments, the engineered hFVIII polypeptide comprises amino acid sequence of SEQ ID NO: 3, SEQ ID NO: 4, SEQ ID NO: 5, or SEQ ID NO: 6.

In a second aspect, the present invention provides an isolated nucleic acid fragment encoding an engineered hFVIII polypeptide disclosed herein.

In a third aspect, the present invention provides an expression vector, which include a nucleic acid fragment disclosed herein operably linked to a promoter.

In a fourth aspect, the present invention provides a recombinant AAV (rAAV) vector, which include a nucleic acid fragment disclosed herein operably linked to a promoter.

In a fifth aspect, the present invention provides a pharmaceutical composition, which includes an expression vector disclosed herein or a rAAV vector disclosed herein.

In a sixth aspect, the present invention provides a method for treating a hemophilia A patient. The method includes administering to the patient an effective amount of a pharmaceutical composition disclosed herein.

These and other features, aspects, and advantages of the present invention will become better understood with reference to the following description and appended claims.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 shows the diagrams of human wild type FVIII and FVIII-SQ.

FIGS. 2A-2C show the results of coagulation time of hybrid FVIIIs. FIG. 2A is the comparison of the activities between hHC and mHC; FIG. 2B is the comparison of the activities between hHC and dHC; FIG. 2C is the comparison of the activities between hHC and maHC.

FIG. 3A shows that the A1 and A2 domains of human and megabat FVIII were mixed and matched to construct more hybrid FVIIIs, M1H2 and H1M2. FIG. 3B shows coagulation time of various FVIII proteins.

FIG. 4 shows that the A1 domain of the heavy chain can be subdivided into D1 and D2 regions, and the A2 domain into D3 and D4 regions.

FIG. 5A are diagrams showing that the D1 or D4 domains of megabat FVIII was replaced with its human counterpart to construct hybrid megabat FVIIIs: hD1 and hD4. FIG. 5B shows coagulation time of various FVIII proteins.

FIG. 6A are diagrams showing that various D1-D4 regions of human FVIII were replaced by their counterparts in megabat FVIII to construct hybrid human FVIIIs: mD1mD3, mD2, mD3, and mD4. FIG. 6B shows coagulation time of various FVIII proteins.

FIG. 7 shows the sequence alignment of the D1 region of human and megabat FVIII.

FIG. 8A are the ELISA results, which show that the mutations of 8 amino acids, namely, V51L, T62I, E6D, I130F, D133G, E140Q, P153L, and F160L, led to the lower FVIII protein expression levels. FIG. 8B are the aPTT results, which show that the mutations of 8 amino acids, namely, V51L, T62I, E116D, I130F, D133G, E140Q, P153L, and F160L had lower coagulation activities.

FIG. 9 shows the coagulation activities of various mutant FVIIIs.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Replacement therapy to treat hemophilia A may generate inhibitors of the injected or acquired factor VIII, leading to the failure of this replacement therapy.

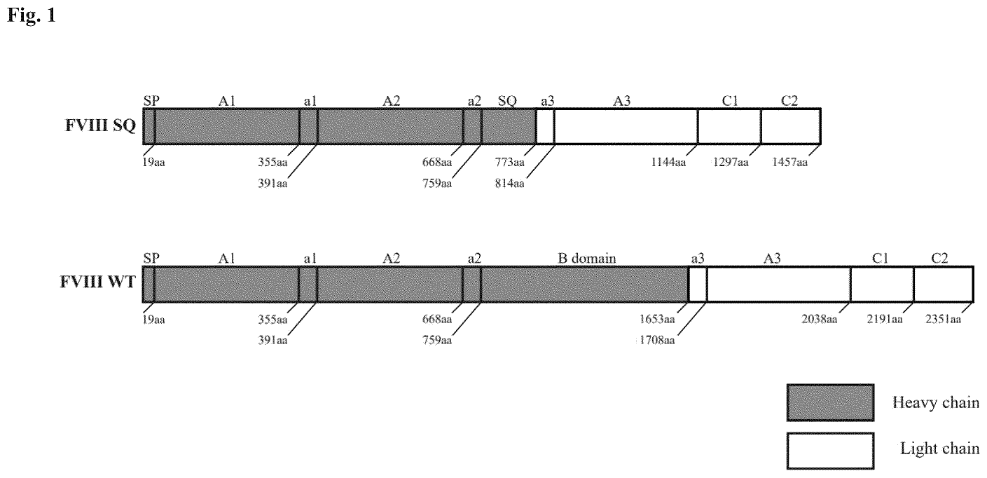

An alternative therapy for hemophilia A is gene therapy based on rAAV vectors. The rAAV vectors allow long-term, stable expression of transgenes in vivo for therapeutic purposes. The coding region of F8 is 7035 bp long and can be divided into 6 domains, namely. A1, A2, B, A3, C1, C2 (FIG. 1, lower panel). For rAAV vectors to be efficiently packaged into adeno-associated virus (AAV) capsids, the size of the rAAV vector that includes the expression cassette of a therapeutic gene and two ITRs is around 5 kb.

Due to the limitation of AAV packaging capacity, the full-length F8 coding region inserted into rAAV vectors cannot be packaged into AAV capsids. To address this problem, the coding region of F8 gene needs to be decreased. Previous studies have shown that B domain of FVIII (908 aa) can be replaced with SQ domain (14 aa) while retains the coagulation effect of the full length FVIII. This engineered FVIII is called FVIII-SQ and has 6 domains: A1, A2, SQ, A3, C1, and C2, A1, A2, and SQ domains form the heavy chain and A3, C1, C2 form the light chain of FVIII-SQ. The nucleotide encoding the FVIII-SQ is 4371 bp (FIG. 1, upper panel) so that it can be inserted into rAAV vectors for efficient packaging into AAV capsids.

However, even when the expression cassette is around 5 kb, one key limitation of HA rAAV gene therapy is inefficient secretion of FVIII, likely caused by a slow folding process of the factor in the endoplasmic reticulum. To compensate that, a large amount of rAAV vectors need to be injected to HA patients to make enough functional FVIII. And a large amount of injected rAAV vectors may lead to adverse immune response to the patients.

Since FVIII is a secretion protein, to reduce the required dose of viral vector to tolerable levels, one strategy is to increase the secretion activity of FVIII generated by rAAV vectors by modifying the amino acids of FVIII. More secreted FVIII, higher total clotting activity of FVIII. It has been found that the secretion capacity of porcine FVIII is 10-100-fold higher than human FVIII, and the heavy chain of porcine Factor VIII is responsible for this enhanced secretion (Identification of Porcine Coagulation Factor VIII Domains Responsible for High Level Expression via Enhanced Secretion. JBC, 279, 6546-6552). Thus, the inventors of this application have developed a theory, but not bound by such a theory, that the heavy chain of human FVIII could be engineered to enhance its secretion for use in rAAV gene therapy.

In a first aspect, the present invention provides an engineered human factor VIII (hFVIII) polypeptide comprising at least two substituted amino acids in A1 domain of hFVIII.

As used herein, “engineered” refers to modification by manipulation of genetic material, chemical synthesis, or using other ways to change a protein from its wildtype state to another state. Depending on the context, an engineered FVIII may be called a mutant FVIII, a hybrid FVIII, or a FVIII mutant.

As used herein, “substitute” or “substitution” refers to amino acid replacement where a change from one amino acid to a different amino acid in a protein due to point mutation(s) in the corresponding DNA sequence. A “substituted amino acid” refers to the new amino acid, which has replaced the existing amino acid.

As used herein, “domain” is defined by a continuous sequence of amino acids characterized by e.g., internal amino acid sequence identity to structurally related domains and by sites of proteolytic cleavage by thrombin. A human wild type FVIII containing A1, A2, B, A3, C1, and C2 domains is shown in FIG. 1 lower panel.

To identify a smaller region or amino acids of A1 domain that are responsible for the enhanced secretion capacity, the A1 domain of the human FVIII has been subdivided into D1 and D2 regions.

The amino acid sequence of the human D1 region is set forth in SEQ ID NO: 1 as follows:

ATRRYYLGAVELSWDYMQSDLGELPVDARFPPRVPKSFPFNTSVVY
KKTLFVEFTDHLFNIAKPRPPWMGLLGPTIQAEVYDTVVITLKNMAS
HPVSLHAVGVSYWKASEGAEYDDQTSQREKEDDKVFPGGSHTYV
WQVLKENGPMASDPLCLTYSYLSHVDLVKDLNSGLIGALLVCREGS
LAKE

In some embodiments, the substituted amino acids include L50 and L152 in the A1 domain. Here, L50 refers to the Leucine (L) at position number 50 with respect to SEQ ID NO: 1 has been substituted by other amino acid that is not specified, and L152 refers to the Leucine (L) at position number 152 with respect to SEQ ID NO: 1 has been substituted by other amino acid that is not specified.

In some embodiments, the substituted amino acids include L50V and L152P in the A1 domain. Here, L50V refers to the Leucine (L) at position number 50 with respect to SEQ ID NO: 1 has been substituted by amino acid Valine (V), and L152P refers to the Leucine (L) at position number 152 with respect to SEQ ID NO: 1 has been substituted by amino acid Proline (P).

In some embodiments, the substituted amino acids further include one or more of amino acid substitutions selected from the group consisting of D20, G22, I61, A115, F129. G132, Q139, and L159 in the A1 domain.

In some embodiments, the amino acid substitutions of D20, G22, I61, D115, F129, G132, Q139, and L159 are D20S, G22L, I61T, D115E, F129I, G132D, Q139E, and L159F, respectively.

In some embodiments, the substituted amino acids comprise D20S, L50V, and L152P.

In some embodiments, the substituted amino acids comprise D20S, G22L, L50V, and L152P.

In some embodiments, the engineered hFVIII polypeptide comprises amino acid sequence of SEQ ID NO: 3. SEQ ID NO: 4, SEQ ID NO: 5, or SEQ ID NO: 6.

SEQ ID NO: 3:
ATRRYYLGAVELSWDYMQSSLLELPVDARFPPRVPKSFPFNTSVVYK
KTVFVEFTDHLFNIAKPRPPWMGLLGPTIQAEVYDTVVITLKNMASHP
VSLHAVGVSYWKASEGAEYDDQTSQREKEDDKVFPGGSHTYVWQV
LKENGPMASDPPCLTYSYLSHVDLVKDLNSGLIGALLVCREGSLAKE
KTQTLHKFILLFAVFDEGKSWHSETKNSLMQDRDAASARAWPKMHT
VNGYVNRSLPGLIGCHRKSVYWHVIGMGTTPEVHSIFLEGHTFLVRN
HRQASLEISPITFLTAQTLLMDLGQFLLFCHISSHQHDGMEAYVKVDS
CPEEPQLR
SEQ ID NO: 4:
ATRRYYLGAVELSWDYMQSSLGELPVDARFPPRVPKSFPFNTSVVYK
KTVFVEFTDHLFNIAKPRPPWMGLLGPTIQAEVYDTVVITLKNMASHP
VSLHAVGVSYWKASEGAEYDDQTSQREKEDDKVFPGGSHTYVWQV
LKENGPMASDPPCLTYSYLSHVDLVKDLNSGLIGALLVCREGSLAKE
KTQTLHKFILLFAVFDEGKSWHSETKNSLMQDRDAASARAWPKMHT
VNGYVNRSLPGLIGCHRKSVYWHVIGMGTTPEVHSIFLEGHTFLVRN
HRQASLEISPITFLTAQTLLMDLGQFLLFCHISSHQHDGMEAYVKVDS
CPEEPQLR
SEQ ID NO: 5:
MQIELSTCFFLCLLRFCFSATRRYYLGAVELSWDYMQSSLLELPVDAR
FPPRVPKSFPFNTSVVYKKTVFVEFTDHLFNIAKPRPPWMGLLGPTIQA
EVYDTVVITLKNMASHPVSLHAVGVSYWKASEGAEYDDQTSQREKE
DDKVFPGGSHTYVWQVLKENGPMASDPPCLTYSYLSHVDLVKDLNS
GLIGALLVCREGSLAKEKTQTLHKFILLFAVFDEGKSWHSETKNSLMQ
DRDAASARAWPKMHTVNGYVNRSLPGLIGCHRKSVYWHVIGMGTTP
EVHSIFLEGHTFLVRNHRQASLEISPITFLTAQTLLMDLGQFLLFCHISS
HQHDGMEAYVKVDSCPEEPQLRMKNNEEAEDYDDDLTDSEMDVVR
FDDDNSPSFIQIRSVAKKHPKTWVHYIAAEEEDWDYAPLVLAPDDRS
YKSQYLNNGPQRIGRKYKKVRFMAYTDETFKTREAIQHESGILGPLLY
GEVGDTLLIIFKNQASRPYNIYPHGITDVRPLYSRRLPKGVKHLKDFPIL
PGEIFKYKWTVTVEDGPTKSDPRCLTRYYSSFVNMERDLASGLIGPLLI
CYKESVDQRGNQIMSDKRNVILFSVFDENRSWYLTENIQRFLPNPAGV
QLEDPEFQASNIMHSINGYVFDSLQLSVCLHEVAYWYILSIGAQTDFLS
VFFSGYTFKHKMVYEDTLTLFPFSGETVFMSMENPGLWILGCHNSDF
RNRGMTALLKVSSCDKNTGDYYEDSYEDISAYLLSKNNAIEPRSFSQN
PPVLKRHQREITRTTLQSDQEEIDYDDTISVEMKKEDFDIYDEDENQSP
RSFQKKTRHYFIAAVERLWDYGMSSSPHVLRNRAQSGSVPQFKKVVF
QEFTDGSFTQPLYRGELNEHLGLLGPYIRAEVEDNIMVTFRNQASRPY
SFYSSLISYEEDQRQGAEPRKNFVKPNETKTYFWKVQHHMAPTKDEF
DCKAWAYFSDVDLEKDVHSGLIGPLLVCHTNTLNPAHGRQVTVQEFA
LFFTIFDETKSWYFTENMERNCRAPCNIQMEDPTFKENYRFHAINGYI
MDTLPGLVMAQDQRIRWYLLSMGSNENIHSIHFSGHVFTVRKKEEYK
MALYNLYPGVFETVEMLPSKAGIWRVECLIGEHLHAGMSTLFLVYSN
KCQTPLGMASGHIRDFQITASGQYGQWAPKLARLHYSGSINAWSTKE
PFSWIKVDLLAPMIIHGIKTQGARQKFSSLYISQFIIMYSLDGKKWQTY
RGNSTGTLMVFFGNVDSSGIKHNIFNPPIIARYIRLHPTHYSIRSTLRME
LMGCDLNSCSMPLGMESKAISDAQITASSYFTNMFATWSPSKARLHL
QGRSNAWRPQVNNPKEWLQVDFQKTMKVTGVTTQGVKSLLTSMYV
KEFLISSSQDGHQWTLFFQNGKVKVFQGNQDSFTPVVNSLDPPLLTRY
LRIHPQSWVHQIALRMEVLGCEAQDLY
SEQ ID NO: 6:
MQIELSTCFFLCLLRFCFSATRRYYLGAVELSWDYMQSSLGELPVDAR
FPPRVPKSFPFNTSVVYKKTVFVEFTDHLFNIAKPRPPWMGLLGPTIQA
EVYDTVVITLKNMASHPVSLHAVGVSYWKASEGAEYDDQTSQREKE
DDKVFPGGSHTYVWQVLKENGPMASDPPCLTYSYLSHVDLVKDLNS
GLIGALLVCREGSLAKEKTQTLHKFILLFAVFDEGKSWHSETKNSLMQ
DRDAASARAWPKMHTVNGYVNRSLPGLIGCHRKSVYWHVIGMGTTP
EVHSIFLEGHTFLVRNHRQASLEISPITFLTAQTLLMDLGQFLLFCHISS
HQHDGMEAYVKVDSCPEEPQLRMKNNEEAEDYDDDLTDSEMDVVR
FDDDNSPSFIQIRSVAKKHPKTWVHYIAAEEEDWDY APLVLAPDDRS
YKSQYLNNGPQRIGRKYKKVRFMAYTDETFKTREAIQHESGILGPLLY
GEVGDTLLIIFKNQASRPYNIYPHGITDVRPLYSRRLPKGVKHLKDFPIL
PGEIFKYKWTVTVEDGPTKSDPRCLTRYYSSFVNMERDLASGLIGPLLI
CYKESVDQRGNQIMSDKRNVILFSVFDENRSWYLTENIQRFLPNPAGV
QLEDPEFQASNIMHSINGYVFDSLQLSVCLHEVAYWYILSIGAQTDFLS
VFFSGYTFKHKMVYEDTLTLFPFSGETVFMSMENPGLWILGCHNSDF
RNRGMTALLKVSSCDKNTGDYYEDSYEDISAYLLSKNNAIEPRSFSQN
PPVLKRHQREITRTTLQSDQEEIDYDDTISVEMKKEDFDIYDEDENQSP
RSFQKKTRHYFIAAVERLWDYGMSSSPHVLRNRAQSGSVPQFKKVVF
QEFTDGSFTQPLYRGELNEHLGLLGPYIRAEVEDNIMVTERNQASRPY
SFYSSLISYEEDQRQGAEPRKNFVKPNETKTYFWKVQHHMAPTKDEF
DCKAWAYFSDVDLEKDVHSGLIGPLLVCHTNTLNPAHGRQVTVQEFA
LFFTIFDETKSWYFTENMERNCRAPCNIQMEDPTFKENYRFHAINGYI
MDTLPGLVMAQDQRIRWYLLSMGSNENIHSIHFSGHVFTVRKKEEYK
MALYNLYPGVFETVEMLPSKAGIWRVECLIGEHLHAGMSTLFLVYSN
KCQTPLGMASGHIRDFQITASGQYGQWAPKLARLHYSGSINAWSTKE
PFSWIKVDLLAPMIIHGIKTQGARQKFSSLYISQFIIMYSLDGKKWQTY
RGNSTGTLMVFFGNVDSSGIKHNIFNPPIIARYIRLHPTHYSIRSTLRME
LMGCDLNSCSMPLGMESKAISDAQITASSYFTNMFATWSPSKARLHL
QGRSNAWRPQVNNPKEWLQVDFQKTMKVTGVTTQGVKSLLTSMYV
KEFLISSSQDGHQWTLFFQNGKVKVFQGNQDSFTPVVNSLDPPLLTRY
LRIHPQSWVHQIALRMEVLGCEAQDLY

In a second aspect, the present invention provides an isolated nucleic acid fragment encoding an engineered hFVIII polypeptide disclosed herein. The isolated nucleic acid fragments include all possible nucleic acid sequences encoding the breadth of substitution mutants described herein. All possible nucleic acid sequences consider, but not limited, the principle of degeneracy of codons.

In a third aspect, the present invention provides an expression vector, which include a nucleic acid fragment disclosed herein operably linked to a promoter.

The term “operably linked” means that the regulatory sequences necessary for expression of a coding sequence are placed in the DNA molecule in the appropriate positions relative to the coding sequence so as to effect expression of the coding sequence.

In a fourth aspect, the present invention provides a recombinant AAV (rAAV) vector, which include a nucleic acid fragment disclosed herein operably linked to a promoter.

Human adeno-associated virus (AAV) is a non-pathogenic parvovirus that only productively replicates in cells co-infected by a helper virus, usually adenovirus or herpes virus. The virus has a wide host range and can productively infect many cell types from a variety of animal species. Nevertheless, AAV has not been implicated in any human or animal disease.

AAV binds to cells via a heparan sulfate proteoglycan receptor. Once attached. AAV entry is dependent upon the presence of a co-receptor, either the fibroblast growth factor receptor or αvβ5 integrin molecule. In infected cells, the incoming AAV single-stranded DNA (ssDNA) is converted to double-stranded transcriptional template. Cells infected with AAV and a helper virus will undergo productive replication of AAV prior to cell lysis, which is induced by the helper virus rather than AAV. Helper virus encodes proteins or RNA transcripts which are transcriptional regulators and are involved in DNA replication or modify the cellular environment in order to permit efficient viral production.

Recombinant AAV (rAAV) vectors are typically produced by replacing the viral coding sequences with transgenes of interest. These vectors have been shown to be highly efficient for gene transfer and expression at a number of different sites in vitro and in vivo. They have consistently mediated stable expression and have been safe in studies performed in the respiratory tract, the central nervous system, skeletal muscle, liver, and eye. The efficiency of rAAV-mediated transduction has increased as the titer and purity of rAAV preparations has improved.

The inverted terminal repeats (ITRs) from the AAV genome are the only viral sequences required in cis to generate rAAV vectors. Recombinant constructs containing two ITRs bracketing a gene expression cassette of ˜5 kb are converted into a ssDNA vector genome and packaged into AAV particles in the presence of AAV rep and cap gene products and helper functions. Methods or production and purification of rAAV are known in the art.

In the rAAV vectors, the nucleic acid fragment encoding an engineered factor VIII disclosed herein is less than 5 kb and it has been inserted into an expression cassette flanked by two ITRs to achieve efficient packaging into AAV particles.

In either the expression vectors or the rAAV vectors, the nucleic acid sequence encoding the engineered factor VIII disclosed herein is operably linked to a promoter. The promoter can be, but is not limited to, a constitutive promoter, an inducible promoter, a liver-specific promoter, a hepatocyte-specific promoter, or a synthetic promoter.

Constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter.

An inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline.

A liver-specific promoter can be, but is not limited to, an albumin promoter, an alpha-1-antitrypsin promoter, or a hepatitis B virus core protein promoter.

A synthetic promoter may comprise, for example, regions of known promoters, regulatory elements, transcription factor binding sites, enhancer elements, repressor elements, and the like. For example, a synthetic promoter can comprise a natural promoter and a combination of enhancers from transcription factors. To achieve appropriate expression levels of the nucleic acid, protein, or polypeptide of interest, any of a number of promoters suitable for use in the selected host cell may be employed.

In a fifth aspect, the present invention provides a pharmaceutical composition, which includes an expression vector disclosed herein or a rAAV vector disclosed herein.

The term “pharmaceutical composition” refers to a mixture of the expression vectors disclosed herein or the rAAV vectors disclosed herein with other chemical components, such as diluents or carriers. The pharmaceutical composition facilitates administration of the compound to an organism. Pharmaceutical compositions will generally be tailored to the specific intended route of administration. A pharmaceutical composition is suitable for human and/or veterinary applications.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen.

In a sixth aspect, the present invention provides a method for treating a hemophilia A patient. The method includes administering to the patient an effective amount of a pharmaceutical composition disclosed herein.

The term “effective amount” or “therapeutically effective amount” refers to the quantity of a composition, for example a composition comprising rAAV vectors, that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

One of skill in the art recognizes that an amount may be considered therapeutically “effective” even if the condition is not totally eradicated or prevented, but it or its symptoms and/or effects are improved or alleviated partially in the subject. Various indicators for determining the effectiveness of a method for treating hemophilia A patient are known to those skilled in the art.

As used herein, the terms “treating,” “treatment,” “therapeutic,” or “therapy” do not necessarily mean total cure or abolition of the disease or condition Amy alleviation of any undesired signs or symptoms of a disease or condition, to any extent can be considered treatment and/or therapy. Furthermore, treatment may include acts that may worsen the patient's overall feeling of well-being or appearance.

Other ingredients may be included in the claimed composition, such as other active agents, preservatives, buffering agents, salts, a pharmaceutically acceptable carrier, or other pharmaceutically acceptable ingredients.

As used herein, a “carrier” refers to a compound that facilitates the incorporation of a compound into cells or tissues. For example, without limitation, dimethyl sulfoxide (DMSO), Ethanol (EtOH), or PEG400 is a commonly utilized carrier that facilitates the uptake of many organic compounds into cells or tissues of a subject.

### Definitions

Unless defined otherwise, all technical and scientific terms used herein have the same meaning as is commonly understood by one of ordinary skill in the art. In the event that there are a plurality of definitions for a term herein, those in this section prevail unless stated otherwise.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “individual,” “patient,” or “subject” are used interchangeably. None of the terms require or are limited to situation characterized by the supervision (e.g. constant or intermittent) of a health care worker (e.g a doctor, a registered nurse, a nurse practitioner, a physician's assistant, an orderly, or a hospice worker).

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

The term “promoter region” or “promoter” refers to a region of DNA that directs/initiates transcription of a nucleic acid (e.g, a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell. For example, a liver-specific promoter is a promoter that directs/initiates transcription in liver tissue to a substantially greater extent than other tissue types.

Enhancer: A nucleic acid sequence that increases the rate of transcription by increasing the activity of a promoter.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. This definition is also sometimes applied to the arrangement of nucleic acid sequences of a first and a second nucleic acid molecule wherein a hybrid nucleic acid molecule is generated.

The term “nucleotide,” as used herein, generally refers to a base-sugar-phosphate combination A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2,7-dimethoxy-4,5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, |dR110] ddCTP, [dTAMRA] ddGTP, and [dROX] ddTTP available from Perkin Elmer, Foster City. Calif: FluoroLinkDeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rhodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP. Cascade Blue-7-UTP. Cascade Blue-7-dUTP, fluorescein-12-UTP, fluorescein-12-dUTP, Oregon Green 488-5-dUTP, Rhodanine Green-5-UTP. Rhodamine Green-5-dUTP, tetramethylrhodamine-6-UTP, tetramethylrhodamine-6-dUTP, Texas Red-5-UTP. Texas Red-5-dUTP, and Texas Red-12-dUTP available from Molecular Probes. Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

cDNA (complementary DNA): A piece of DNA lacking internal, non-coding segments (introns) and regulatory sequences that determine transcription, cDNA is synthesized in the laboratory by reverse transcription from messenger RNA extracted from cells, cDNA can also contain untranslated regions (UTRs) that are responsible for translational control in the corresponding RNA molecule.

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Transcription factor (TF): A protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

A “corresponding” nucleic acid or amino acid or sequence of either, as used herein, is one present at a site in a factor VIII or fragment thereof that has the same structure and/or function as a site in the factor VIII molecule of another species, although the nucleic acid or amino acid number may not be identical.

Control: A control is an individual or a group of samples used as a standard of comparison for checking the results of a survey or experiment. In some context, a control is expressed as a reference.

“Subunits” of human or animal factor VIII, as used herein, are the heavy and light chains of the protein. The heavy chain of factor VIII contains three domains, A1, A2, and B. The light chain of factor VIII also contains three domains, A3, C1, and C2.

“Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. For example, a “positive effect” may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject an amount of cells that is “therapeutically effective.” The term “therapeutically effective” should be understood to have a definition corresponding to ‘having a therapeutic effect.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Methods and Materials

### Plasmid Construction

To generate the plasmids containing heavy chain mutants of FVIII, the plasmid pAAV-CB-FVIII-SQ was used as the backbone. This plasmid contains CB promoter and F8 gene encoding human FVIII-SQ. CB promoter is consisted of a cytomegalovirus enhancer and a human beta actin promoter. The inserts of heavy chain mutants were obtained by PCR amplifications where human and megabat F8 genes were used as the templates. Primers were synthesized by Tsingke Biological Technology. The backbone and the designed inserts were digested with Not I-HF and Kpn I-HF (NEB) and then purified by E.Z.N.A. Gel Extraction Kit (Omega Bio-Tek). The sticky ligations of the inserts with the backbone generated the plasmids with various heavy chain mutants.

### Tissue Culture and Transfection

HEK293 cells were cultured in Dulbecco's modified Eagle's medium (Invitrogen) supplemented with 10% fetal bovine serum (Gibco), 100 μg of penicillin/ml and 100 U of streptomycin/ml. For transfection, 0.75 ug of the FVIII plasmid was mixed with 2.25 μl of PolyJet (SignaGen laboratories) and added to each well of a 12-well plate according to the manufacturer's instruction. The media were changed to Ham's F12 media (Corning) supplied with 2% heat-inactivated fetal bovine serum at 6 hours after transfection. Media were collected for aPTT assay at 24 hours after the media change.

aPTT

For in vitro assay, Refacto (Genetics Institute, Cambridge, MA) was serially diluted with media from 1 U/ml (200 ng/ml) by 1/2's till 1/512 and used as the standards. Media collected from the transfected mammalian cells were centrifuged at 13,000 rpm for 1 minute and used as the samples. 50 μl of each of STA-PTT reagent, FVIII deficient plasma and sample (or the diluted standards) were mixed in strips of STAGO cuvettes. The mixtures were incubated at 37° C. for 170 seconds. Coagulation time was then initiated and measured by adding 50 μl of 25 mM CaCl₂ using STAGO machine. The FVIII activities were measured according to the standard curve.

### ELISA

Refacto was used as the standards and was two-fold serially diluted using growth media. The media were collected as described above. Mouse plasma samples were prepared in HEPES buffer at a 1:10 dilution. For ELISA, 96-well plates were coated with the capture antibody (PAH-FVIII-S, 7.1 mg/ml, 1:2000) in coating buffer (0.1 M sodium bicarbonate and carbonate, pH 9.6) at 4° C. overnight. The wells were blocked with 3% BSA in PBST buffer (140 mM NaCl, 2.5 mM KCl, 8 mM Na₂HPO₄, 2 mM KH₂PO₄ and 0.05% Tween 20, pH 8.4) at room temperature for 1 hour. After washing with PBST buffer for three times, 100 μl of the standards and samples were added and incubated at room temperature for 1 hour. After the plates were washed three times with PBST buffer, the detecting antibody (GMA-8021-HRP, 1:200) was added. The plates were incubated at room temperature for 1 hour and were washed three times with PBST buffer. The color was developed using 1× SureBlue TMB 1-Component Microwell Peroxidase Substrate. The color development was carried out at room temperature for 1-10 min and stopped by adding 0.5M H₂SO₄. OD values were determined by a spectrophotometer at 450 nm and FVIII amounts were calculated according to the standard curve.

### Example 1. Identification of A1 Domain of the FVIII Heavy Chain of Megabat with Enhanced Secretion Activity

Previous study has found that the secretion capacity of porcine FVIII is 10-100-fold higher than human FVIII, and the heavy chain of porcine Factor VIII is responsible for this enhanced secretion (Identification of Porcine Coagulation Factor VIII Domains Responsible for High Level Expression via Enhanced Secretion. JBC, 279, 6546-6552). The inventors have hypothesized that FVIII in other animals might also have enhanced secretion and have designed studies to pinpoint the heavy chain region that responsible for the enhanced secretion.

To this purpose, F8 gene nucleotide sequences from various animals are aligned. FVIIIs from monkey, megabat and dolphin were chosen because monkeys jump on land, megabats fly in the sky and dolphin swing in water. The heavy chain sequence of FVIII from these animals were fused with the human FVIII light chain sequence to form hybrid FVIIIs. Plasmids containing the expression cassettes of these hybrid FVIIIs were transfected into cells. 24 hours post transfection, the culture supernatant was collected for partial thromboplastin time (aPTT) test to measure the coagulation activity of FVIII.

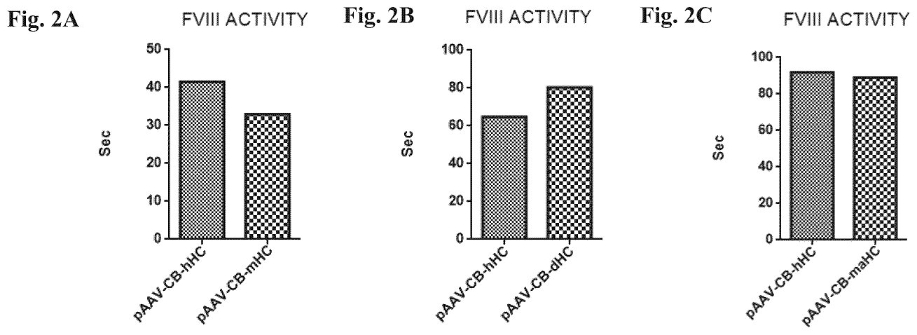

FIGS. 2A-2C show the results of aPPT.mHC is the hybrid FVIII that is consisted of the heavy chain of megabat FVIII and the light chain of human FVIII, dHC is the hybrid FVIII that is consisted of the heavy chain of dolphin FVIII and the light chain of human FVIII, maHC is the hybrid FVIII that is consisted of the heavy chain of monkey FVIII and the light chain of human FVIII. In each of FIGS. 2A-2C, Human FVIII (hHC, refers to FVIII-SQ) was used as the control. FIG. 2A is the comparison of the activities between hHC and mHC: FIG. 2B is the comparison of the activities between hHC and dHC; FIG. 2C is the comparison of the activities between hHC and maHC.

As shown in FIGS. 2A-2C, the hybrid FVIII protein mHC that contains the heavy chain of megabat FVIII and light chain of human FVIII had the shortest clotting time, indicating that its secretion level was the highest, which led to the highest coagulation activity, mHC is different than the human FVIII is that mHC contains the heavy chain of megabat FVIII. This suggests that the heavy chain of megabat FVIII contributes to the enhanced secretion of the hybrid FVIIImHC.

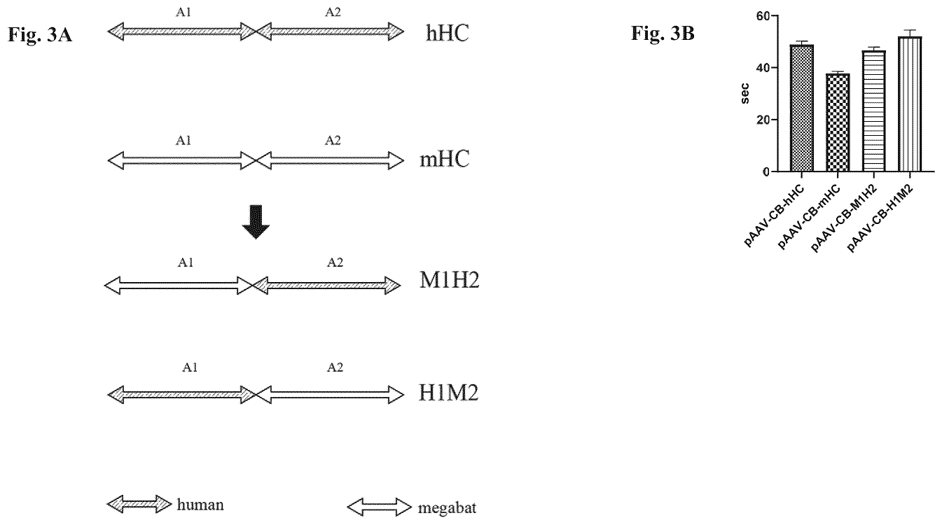

Next, the A1 and A2 domains of human and megabat FVIII were mixed and matched to construct more hybrid FVIIIs, M1H2 and H1M2. M1H2 includes A1 domain of megabat heavy chain (mHC) and A2 domain of human heavy chain (hHC), and H1M2 includes A1 domain of hHC and A2 domain of mHC (FIG. 3A).

As shown in FIG. 3B, comparing to the human FVIII (hHC), M1H2 had a shorter clotting time, indicating that its secretion level was higher, which led to higher coagulation activity. The data suggests that the replacement of A1 domain of hHC with A1 domain of mHC could accelerate coagulation.

### Example 2. Identification of Smaller Region of A1 Domain Linked to Enhanced Secretion Capacity

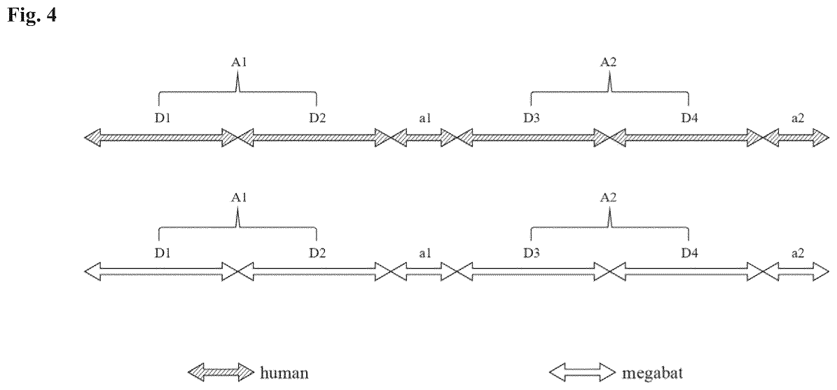

For further mapping, Phymol software was used to predict the structure of the heavy chain of FVIII. According to such prediction, the A1 domain of the heavy chain has been subdivided into D1 and D2, and the A2 domain into D3 and D4 to facilitate further pinpoint the regions in the heavy chain that contributes to the enhanced secretion (FIG. 4).

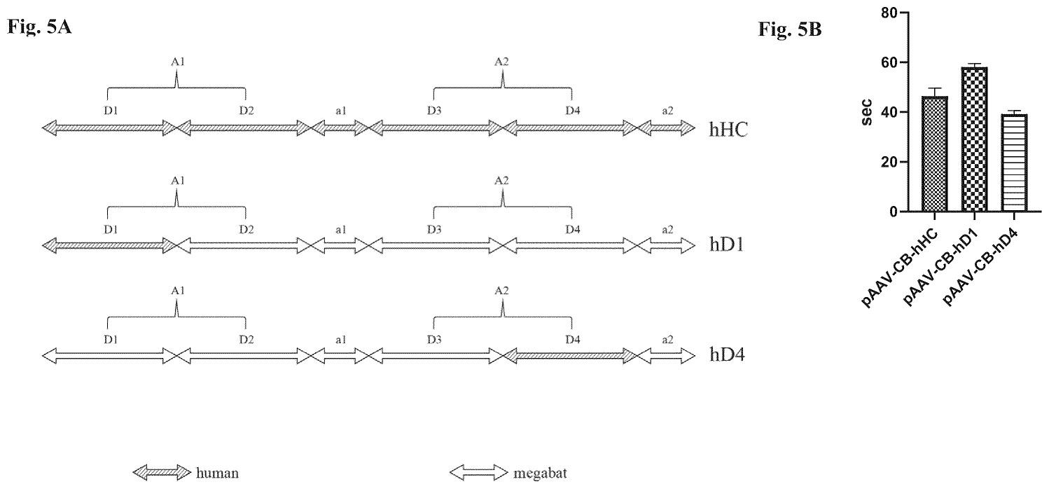

Both negative and positive selection strategies were adopted. In the negative selection strategy, the D1 or D4 domains of megabat FVIII was replaced with its human counterpart to construct hybrid megabat FVIIIs: hD1 and hD4 (FIG. 5A). As shown in FIG. 5A, hD1 contains the D1 region of human FVIII heavy chain (hHC) and the D2-D4 regions of megabat FVIII heavy chain: hD4 contains the D4 region of human FVIII heavy chain (hHC) and the D1-D3 regions of megabat FVIII heavy chain. All the FVIII mutants in the expression cassettes also contain the same light chain and other necessary elements and they are not shown in the figure. Because human FVIII has lower secretion activity, this change would reduce the efficiency of hybrid megabat FVIIIs secretion. Indeed, as shown in FIG. 5B, hD1 had longer clotting time when comparing to human FVIII (hHC). This result suggests that human D1 might decrease the secretion activity of the hybrid megabat FVIII, which in turn led to its decreased coagulation activity. Thus, D1 domain could be important for FVIII secretion.

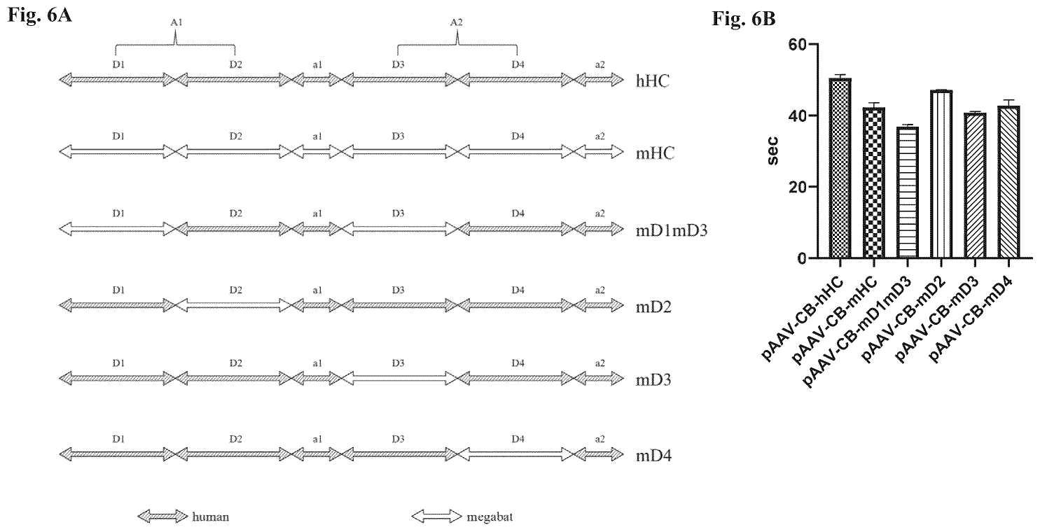

In the positive selection strategy, various D1-D4 regions of human FVIII was replaced by their counterparts in megabat FVIII to construct hybrid human FVIIIs: mD1mD3, mD2, mD3, and mD4 (FIG. 6A). Because megabat FVIII has higher secretion activity, this change would increase the efficiency of hybrid human FVIIIs secretion. As shown in FIG. 6B, all hybrid human FVIIIs showed shorter clotting time than human FVIII hHC), with mD1mD3 having the shortest clotting time. The data suggests that D1 may be the key region responsible for FVIII secretion.

The negative selection and positive selection results suggest that megabat D1 region could be responsible for the high secretion capacity of megabat FVIIII.

### Example 3. Identification of Amino Acids in D1 Region Responsible for Enhanced Secretion

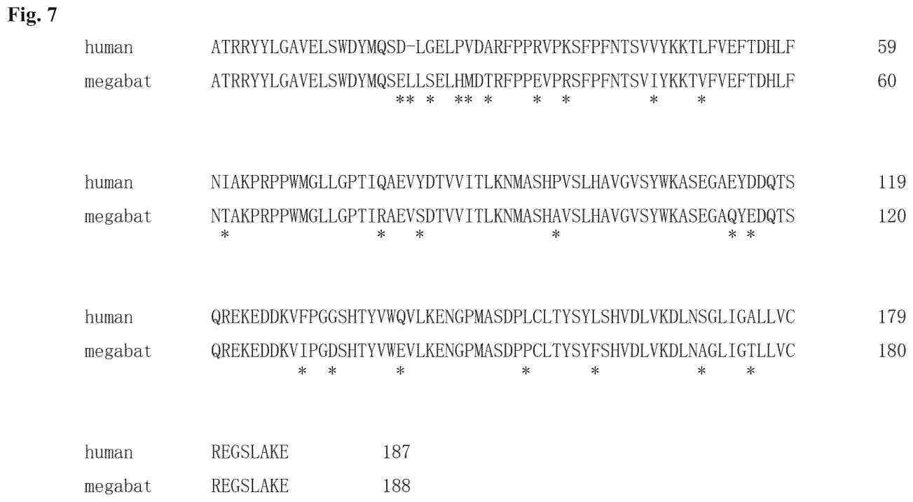

The human D1 (SEQ ID NO: 1) and megabat D1 (SEQ ID NO: 2, see below for its sequence) regions were aligned, and 23 amino acids difference was found (FIG. 7, marked with asterisk). Note that megabat D1 region has one additional amino acid than human D1 region (FIG. 7, “-” in the human sequence represents a missing amino acid). In the following, various mutant megabat FVIIIs and human FVIIIs will be generated. The positions of amino acids in the mutant human FVIIIs are in reference to SEQ ID NO: 1, and the positions of amino acids in the mutant megabat FVIIIs are in reference to SEQ ID NO: 2.

Amino acid sequence of SEQ ID NO: 2 is set forced below:

ATRRYYLGAVELSWDYMQSELLSELHMDTRFPPEVPRSFPFNTSVIYK
KTVFVEFTDHLFNTAKPRPPWMGLLGPTIRAEVSDTVVITLKNMASH
AVSLHAVGVSYWKASEGAQYEDQTSQREKEDDKVIPGDSHTYVWEV
LKENGPMASDPPCLTYSYFSHVDLVKDLNAGLIGTLLVCREGSLAKE

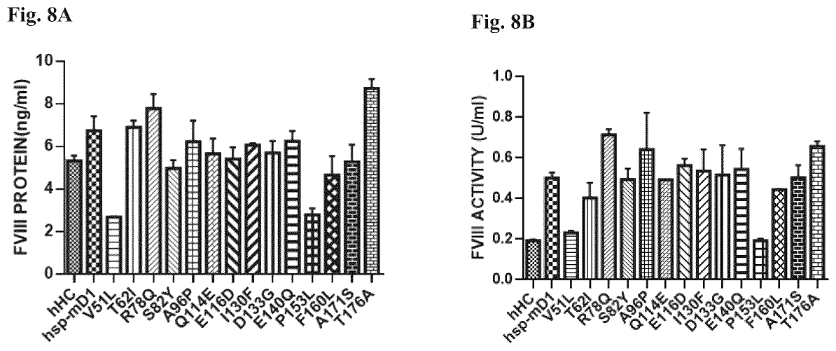

To identify the amino acids that affect FVIII protein secretion, 8 of the 23 different amino acids in megabat D1 were replaced with the corresponding ones from human D1, respectively, to make various mutant FVIIIs: V51L, T62I, E116D, I130F, D133G, E140Q, P153L, and F160L. Note that all the positions of amino acids here are in reference to the megabat sequence SEQ ID NO: 2. For example, V51L here refers to a mutant megabat FVIII where V at position 51 in reference to SEQ ID NO: 2 is changed to L (see FIG. 7). aPTT and ELISA analysis were conducted for the constructed mutant FVIIIs. FIG. 8A are the ELISA results, which show that the mutations of 8 amino acids, namely, V51L, T62I, E116D, I130F, D133G, E140Q, P153L, and F160L, led to the lower FVIII protein expression levels (FIG. 8A). FIG. 8B are the aPTT results, which show that the mutations of 8 amino acids, namely, V51L, T62I, E116D, I130F, D133G, E140Q, P153L, and F160L had lower coagulation activities (FIG. 8B). These data suggest that the 8 amino acids are playing an important role for the enhanced secretion capacity of megabat FVIII. Change the corresponding 8 amino acids in human FVIII might increase the secretion capacity of human FVIII. Due to the additional amino acid in the D1 regions of megabat FVIII (see FIG. 7), mutations of L50V, I61T, D115E, F129I, G132D, Q139E, L152P, and L159F in human FVIII could increase the human FVIII secretion.

Note that two amino acid mutations, V51L and P153L, could reduce the secretion of megabat FVIII the most (FIGS. 8A-8B). In human, mutations of L50V and L152P could be two key amino acid mutants to increase the secretion and coagulation activity of human FVIII.

### Example 4. Identification of Other Amino Acids that Contribute to Enhanced Secretion Activity

At the location from the 20^{th} amino acid to the 23^{rd} amino acid of the megabat D1 region, the megabat sequence is ELLS (FIG. 7); the corresponding amino acid sequence in the human sequence is DLG (FIG. 7). Since FVIII is a secreted protein, it must go through endoplasmic reticulum after it has been released from the synthesis site in the cell. It is hypothesized that D1 may be interacted with the Bip region of the endoplasmic reticulum. It is known that it consumes ATP when FVIII is transported out of a cell. Due to limited storage of ATP in cells, proteins that consume less ATP could be more transported out of cells, thus have more secreted proteins. Based on the analysis of the affinity and hydrophobicity of proteins, the human DLG sequence was changed to the SLG(D20S) or SLL (D20S, G22L). Since these are human FVIII mutants, the amino acid positions are based on SEQ ID NO: 1. For example, G22L here refers to a mutant FIII where G at position 22 in reference to SEQ ID NO: 1 is changed to L.

Human D1 region was mutated to include SLL (D20S, G22L), SLG (D20S), or both, and combined with other amino acid mutations to form various FVIII mutants as listed in Table 1.

| | | Number of |
|---|---|---|
| | | mutated |
| Human FVIII mutants | Mutated amino acids | amino acids |
| pAAV-CB-F8-SLG-3MU-SQ | D20S; L50V; I61T; L152P | 4 |
| pAAV-CB-F8-SLG-2MU-L152-SQ | D20S; L50V; I61T | 3 |
| pAAV-CB-F8-SLL-3MU-SQ | D20S; G22L; L50V; I61T; L152P | 5 |
| pAAV-CB-F8-SLL-2MU-L152-SQ | D20S; G22L; L50V; I61T | 4 |
| pAAV-CB-F8-SLG-2MU-SQ | D20S; L50V; L152P | 3 |
| pAAV-CB-F8-SLL-2MU-SQ | D20S; G22L; L50V; L152P | 4 |

Table 1: Various mutant FVIIIs with DLG sequence mutations. The DLG sequence in the Human D1 region was mutated to SLL (D20S, G22L), or SLG (D20S), or both, and combined with other amino acid mutations to form various mutant FVIIIs with 3-5 amino acid mutations.

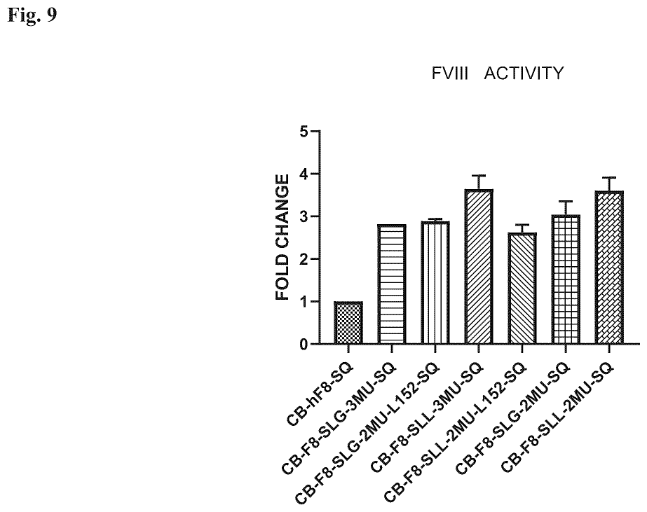

To compare the coagulation activities of mutant FVIIIs to a human FVIII (hF8-SQ), aPTT assay was performed. As shown in FIG. 9, all mutant FVIIIs had higher coagulation activities than hF8-SQ. The data demonstrate that various combination of these mutated amino acids could enhance the secretion of human FVIII.

In summary, a total of 10 mutations in human FVIII, namely, D20S, G22L, L50V, I61T, D115E, F129I, G132D, Q139E, L152P, and L159F have been identified as the key mutations for increasing human FVIII secretion and activity.

### Example 5. Making a Recombinant AAV (rAAV) Vector that Includes an Engineered hFVIII Polypeptide Disclosed Herein

293 suspension cells were seeded in a 3 L bioreactor at 0.8×10⁶ cell/mL. Three plasmids (pAAV-hFVIII, pAd-helper, pRep/Cap) were mixed at a ratio of 1:1:1, and then mixed with PEI at a ratio of 1:2 (1 ug plasmid:2 ul PEI). The mixture was incubated for 15 min at room temperature and then was added to the bioreactor. At 72 hr post transfection, the cells were lysed with lysis buffer containing 1% Tween-20. Then, 50 U/mL Benzonase and 1 mM MgCl₂ were added into the bioreactor and incubated at 37° C. for 3 hr to digest non-packaged cellular, viral, plasmid DNAs and RNAs. The digested cell lysates were clarified and concentrated by filtration. The rAAV vectors were purified by an AAVX affinity column followed by an anionic column. After buffer change, rAAVs were sterile filtered and stored at −80° C.

### Example 6. Making a Pharmaceutical Composition that Includes an Engineered hFVIII Polypeptide Disclosed Herein

The rAAV vectors purified in Example 5 are mixed with other active agents, preservatives, buffering agents, salts, a pharmaceutically acceptable carrier, or other pharmaceutically acceptable ingredients to make a pharmaceutical composition ready to be tested in hemophilia A patients.

### Example 7. Treating a Hemophilia a Patient with a Pharmaceutical Composition that Includes an Engineered hFVIII Polypeptide Disclosed Herein

Patients with hemophilia A are recruited for phase 1 clinic trial. The dosage to be tested will be from 0.5×10¹² Vg/Kg to 6×10¹³ Vg/Kg for the engineered hFVIII polypeptide.

The hemophilia A patients are given intravenously a pharmaceutical composition comprising rAAV vectors encoding an engineered hFVIII polypeptide disclosed herein. The FVIII expression levels will be monitored for 4 years after injection. The first checking point is the 7th day after initial injection. The checking internals are 1-2 weeks for the first 6 months and are 1-3 months thereafter. One year after the initial injection, the patients are found to express >2% normal level of FVIII.

1. An engineered human factor VIII (hFVIII) polypeptide comprising at least two substituted amino acids in A1 domain of hFVIII.

2. The engineered hFVIII polypeptide of claim 1, wherein the substituted amino acids comprise L50 and L152 in the A1 domain.

3. The engineered hFVIII polypeptide of claim 1, wherein the substituted amino acids comprise L50V and L152P in the A1 domain.

4. The engineered hFVIII polypeptide of claim 2, wherein the substituted amino acids further comprise one or more of amino acid substitutions selected from the group consisting of D20, G22, I61, D115, F129, G132, Q139, and L159 in the A1 domain.

5. The engineered hFVIII polypeptide of claim 4, wherein the amino acid substitutions of D20, G22, I61, D115, F129, G132, Q139, and L159 are D20S, G22L, I61T, D115E, F129I, G132D, Q139E, and L159F, respectively.

6. The engineered hFVIII polypeptide of claim 1, wherein the substituted amino acids comprise D20S, L50V, and L152P.

7. The engineered hFVIII polypeptide of claim 1, wherein the substituted amino acids comprise D20S, G22L, L50V, and L152P.

8. The engineered hFVIII polypeptide of claim 1, comprising amino acid sequence of SEQ ID NO: 3, SEQ ID NO: 4, SEQ ID NO: 5, or SEQ ID NO: 6.

9. An isolated nucleic acid fragment encoding the engineered hFVIII polypeptide according to claim 1.

10. (canceled)

11. A recombinant AAV (rAAV) vector comprising the nucleic acid fragment of claim 9, wherein the nucleic acid fragment is operably linked to a promoter.

12. A pharmaceutical composition comprising the rAAV vector of claim 11.

13. A method for treating a hemophilia A patient, comprising administering to the patient an effective amount of a pharmaceutical composition of claim 12.
